(19) United States
(12) Patent Application Publication
(10) Pub. No.: US 2023/0035425 A1
(43) Pub. Date: Feb. 2, 2023

(54) BALLOON CATHETER AND METHODS OF TREATMENT USING SAME

(71) Applicant: Raghavendra Vitthalrao GHUGE, Tyler, TX (US)

(72) Inventor: Raghavendra Vitthalrao GHUGE, Tyler, TX (US)

(21) Appl. No.: 17/815,282

(22) Filed: Jul. 27, 2022

Related U.S. Application Data
(60) Provisional application No. 63/203,748, filed on Jul. 29, 2021.

(51) Int. Cl. A61M 25/10

(52) U.S. Cl. CPC A61M 25/1002; A61M 25/1011; A61M 2025/1059; A61M 2025/109

(57) ABSTRACT

Balloon catheters with an elongate shaft defining a hollow body have an inflatable balloon at a distal end thereof. The balloon has a plurality of internal chambers that are inflatable to differing pressures. When inflated, the balloon has a generally hourglass shape having a neck between a distal end and a proximal end and a port at the neck that is in open communication the hollow body of the shaft and in open communication with an environment external to the balloon. The balloon catheter is inflated in a lumen of a patient to its hourglass shape with its proximal and distal ends in direct contact with normal endothelium juxtaposed to a target lesion with the neck of the balloon at the target lesion. A cutting tool is deployed through the port and an opening having a flap is cut into the target lesion and the plaque is removed thereof.

### RELATED APPLICATIONS

This application claims the benefit of U.S. Provisional Application No. 63/203,748, filed Jul. 29, 2021, the entirety of which is incorporated herein by reference.

### TECHNICAL FIELD

This application relates to balloon catheters, more particularly, to balloon catheters having a plurality of internal chambers inflatable to preselected pressures to produce an hourglass shape and having a port in the neck of the hourglass-shaped balloon.

### BACKGROUND

Catheters are used in various procedures for delivering therapeutic means to a treated site (e.g., body organ or passageway such as blood vessels). In many cases, a small inflatable balloon is guided to the treated site. Once the balloon is in place, it is inflated by the operator for affixing it in place, for expanding a blocked vessel, for placing treatment means (e.g., a stent) and/or for delivering surgical tools (e.g., knives, drills, etc.) to a desired site.

Coronary artery stenosis is a condition where lipid and calcium plaque narrow the lumen of the coronary artery, resulting in blockage and increased risk of heart attack. This disease causes the thickening and narrowing of the coronary artery, the vessel that brings blood to the heart as shown in FIGS. 1A and 1B at reference 10, 20. This can disrupt the flow of oxygen and nutrients to the heart and cause serious problems.

As sequentially illustrated in FIG. 1A, balloon angioplasty is a procedure where the balloon catheter is deployed inside the lumen of the stenotic artery 10, 12 and is inflated 14 to displace the plaque from the stenotic lesion into adjacent normal healthy coronary artery wall and open the stenotic artery 16. The typical balloon is shaped as an elongate body of uniform diameter that tapers distally and proximally, see FIG. 1A at 14. This displaces the plaque to the proximal and distal edges of the balloon once inflated. Another complication of traditional balloon angioplasty is the deformation of artery walls at the site of the stenosis. This is caused by the dilation and expansion of the arterial walls as the balloon squeezes the plaque into the wall causing arterial wall damage (muscularis), which weakens the wall. This also distorts the structure of all the layers of the artery wall (intima, media, and external), making the wall prone to aneurysm, dissection, rupture, and collapse.

Often times, as shown sequentially in FIG. 1B, a stent is deployed after or during balloon angioplasty to prevent collapse of the artery or re-stenosis due to plaque. These two procedures are the current state of the art, but have some risks associated therewith, such as displacement of plaque during the procedure (plaque rupture accompanies 42% of procedures) or injury to adjacent normal arterial wall from the pressure of the balloon or from the placement and expansion of the stent, including the risk of rupture of the arterial wall. Stent angioplasty is known to have an incidence of 38% of re-stenosis, the protrusion of plaque through the stent subsequent to the procedure. Drug eluting stents are used to reduce the occurrence of in-stent stenosis, which not 100% effective, and in fact, pose a risk of a STEMI (ST-segment elevation myocardial infarction, i.e., a classic heart attack) due to the possible rupture of the plaque and release of inflammatory material into the vascular lumen. The use of a stent does nothing to reduce the total plaque burden, but simply spreads the plaque out while most likely damaging the structure of the coronary artery. Once a stent is placed, it can never be fully removed and the artery will always be scarred and weaker.

There is a need for improved procedures and balloon catheters that reduce the risks noted above and/or the re-occurrence of the stenosis, especially one that actually removes the plaque from the artery rather than merely applying pressure thereto.

### SUMMARY

In all aspects, balloon catheters are disclosed that have an elongate shaft having an internal hollow body and a balloon at the distal end of the shaft. The balloon is inflatable and deflatable in accordance with pressure of a fluid supplied to the inside of the balloon through the elongate shaft. The balloon has a plurality of internal chambers that are inflatable to differing pressures, thereby, when inflated, the balloon has a generally hourglass shape having a neck between a distal end and a proximal end of the balloon and has a port at the neck of the balloon in open communication with the internal hollow body and in open communication with an environment external to the balloon. The plurality of internal chambers of the balloon are inflated sequentially or simultaneously and are controllably inflatable. The plurality of internal chambers comprise at least a distal chamber, a neck chamber, and a proximal chamber. The port includes a radially extendable tube fixed to the neck chamber; wherein inflation of the neck chamber extends the tube radially outward. The radially collapsible conduit is juxtaposed to the elongate shaft, radially surrounded by the balloon, and having open distal and proximal ends, when inflated, in fluid communication with the environment.

In one embodiment, the elongate shaft is bifurcated at the distal end into a first hollow body and a second hollow body and comprises a flexible saddle between and joining the first and second hollow bodies. At least a most proximal of the plurality of internal chambers is fully circumferential and is positioned on the elongate shaft prior to the flexible saddle, and the port is positioned in one of the first and second hollow bodies. In this embodiment, the radially collapsible conduit juxtaposed to the elongate shaft that is bifurcated to continue along each of the first and second hollow bodied. The radially collapsible conduit has an open proximal end and first and second open distal ends positioned for fluid communication with the environment. The flexible saddle enables the first and second hollow bodies to separate from one another into a Y-shape as the balloon is inflated. The first balloon portion connected to the first hollow body and the second balloon portion connected to the second hollow body have at least two internal chambers each. The first balloon portion and the second balloon portion are less than fully circumferential.

In all embodiments, the cuff operatively connects the balloon to the elongate shaft. The cuff is rotatable relative to the elongate shaft and is linearly translatable along the elongate shaft. The balloon, in a deflated state, fits within an outer perimeter defined by the cuff.

In another aspect, methods of removing plaque from a lesion in a lumen of a patient are disclosed that include inserting a transportation sheath into the lumen, directing through the transportation sheath a balloon catheter described herein to a target lesion in need of plaque treatment, inflating the balloon of the balloon catheter to its generally hourglass shape with the proximal and distal ends of the balloon in direct contact with normal endothelium proximal and distal to the target lesion and the neck of the balloon at the target lesion, thereby isolating the target lesion from the remainder of the artery, advancing a cutting tool to the target lesion through the port in the balloon catheter, cutting an opening into the endothelium of the target lesion, thereby creating a flap of endothelium, and removing the plaque from inside the target lesion.

The method can include application of additional inflation of the balloon to push plaque toward the flap opening.

The method can include determining the lipid burden of the plaque. This can include application of near-infrared spectroscopy plus intravascular ultrasound or a capacitive micromachines ultrasound transducer.

In one embodiment, the cutting tool is a laser and the opening is a circular cut and the flap has a connection tether of 25 to 45 degrees. The connection tether is at a position of upstream arterial flow, thereby arterial flow holds the flap closed subsequent to the treatment. Removing the plaque includes applying the laser set at a preselected frequency to liquefy the lipid burden present and draining the liquid from the target lesion. The method can also include aspirating inside the target lesion and applying increased pressure to the target lesion by increasing the inflation one or more of the plurality of internal chambers of the balloon.

In another embodiment, a laser deemed appropriate for the lipid burden is placed adjacent to (arterial luminal endothelial surface) the selected site of plaque lesion and activated without entry into the subendothelial space to liquefy the fatty material which then gets released into the subendothelial extracellular matrix. The aspiration catheter is then ultrasonically guided into the subendothelial extracellular matrix space and the liquified fatty material is aspirated. This embodiment avoids cutting a window in the endothelial lining. Optionally, this variation of the method can include administering a drug treatment to one or more of the subendothelial space between the endothelium and muscularis. The drug treatment can include collagen and/or carbon dots comprising stem cells. When collagen is administered, the method includes activating the collagen-carbon dot complex by application of activating wavelength energy.

In all aspects, the method can include displaying an image of the target lesion on a display.

In all aspects, the method includes closing the flap, and, optionally administering a drug treatment to one or more of a sub-endothelium space between the endothelium and muscularis, on an interior surface of the flap, and on an exterior surface of the flap. The drug treatment can include collagen and/or carbon dots comprising stem cells. When collagen is present, the method includes activating the collagen by application of an activating wavelength of energy.

### BRIEF DESCRIPTION OF THE DRAWINGS

FIG. 1A is a cross-sectional, sequential view of a prior art balloon angioplasty.

FIG. 1B is a cross-sectional, sequential view of a prior art stent angioplasty.

FIG. 2 is side, perspective view of a first embodiment of a balloon catheter.

FIG. 3 is a side perspective view of the first embodiment with the balloon in a non-deployed state.

FIG. 4 is a side view showing a second embodiment of a balloon catheter inside a branching artery.

FIG. 5 is a side view inside an artery having the balloon of the balloon catheter of FIG. 2 in a deployed state.

FIG. 6 is a transverse cross-sectional view taken along line 6-6 in FIG. 5.

FIG. 7 is a longitudinal cross-section side view of tools exiting the port of a balloon catheter disclosed herein and positioned inside a stenotic lesion in need of treatment.

FIG. 7 is a cross sectional view of the nanotech delivery catheter (NDC) and the technology contained inside of the NDC

FIG. 8 is a side perspective view of the terminus of a balloon catheter.

FIG. 9 is a flow chart of various methods for removing plaque from an artery.

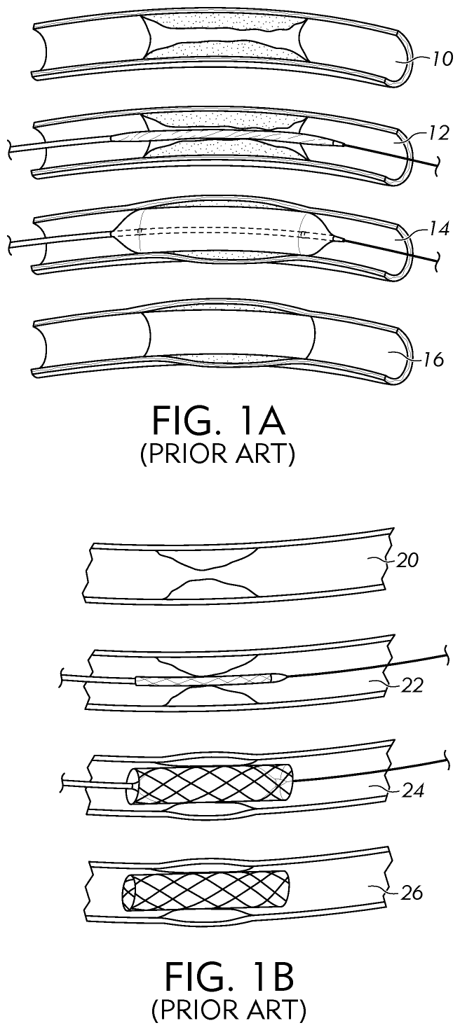

### DETAILED DESCRIPTION

The following detailed description will illustrate the general principles of the invention, examples of which are additionally illustrated in the accompanying drawings. In the drawings, like reference numbers indicate identical or functionally similar elements.

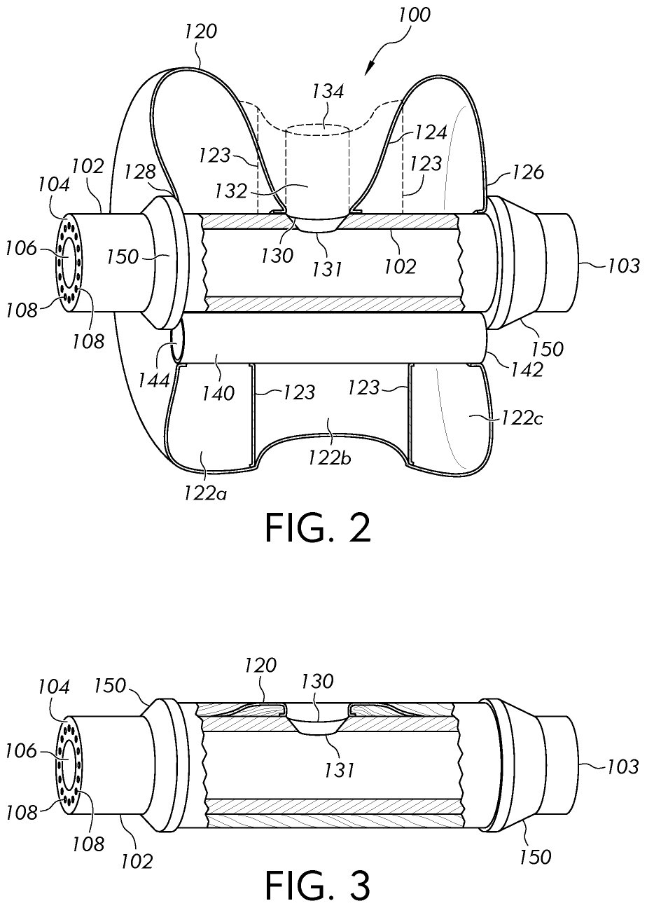

Turning to FIG. 2, a first embodiment of a balloon catheter, generally referred to by the reference number 100, has an elongate shaft 102 that is truncated to show a transverse cross-section thereof and a balloon 120 at a distal end 103 of the elongate shaft that is inflatable and deflatable in accordance with the pressure of a fluid supplied to the inside of the balloon 120 through the internal hollow body 104 of the shaft. The internal hollow body 104 has, running the internal length thereof, a delivery sheath 106 through which any number of tools can be deployed to a target treatment site and at least one fluid delivery tube 108 in fluid communication with the balloon 120. A cuff 150 couples the balloon 120 to the elongate shaft 102. The balloon 120 has a plurality of internal chambers 122 separated from one another by internal walls 123. The plurality of internal chambers 122 are inflatable to differing pressures, thereby, when inflated partially or fully, the balloon has a generally hourglass shape, in three dimensions, having a neck 124 between a distal end 126 and a proximal end 128 of the balloon, and a port 130 at the neck 124 of the balloon 120. The port 130 is in open communication with the delivery sheath 106 housed within the internal hollow body and in open communication with an environment external to the balloon.

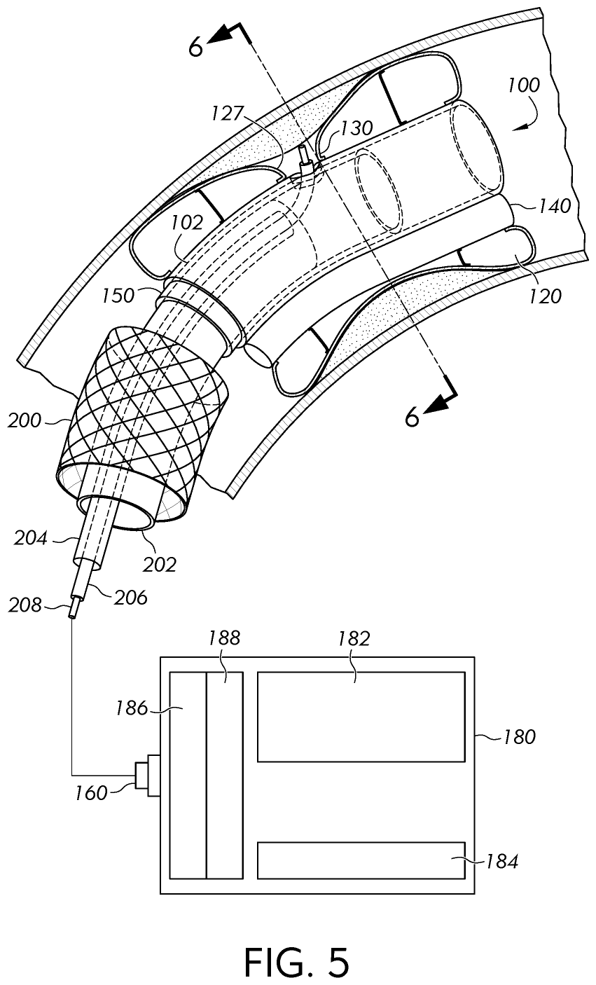

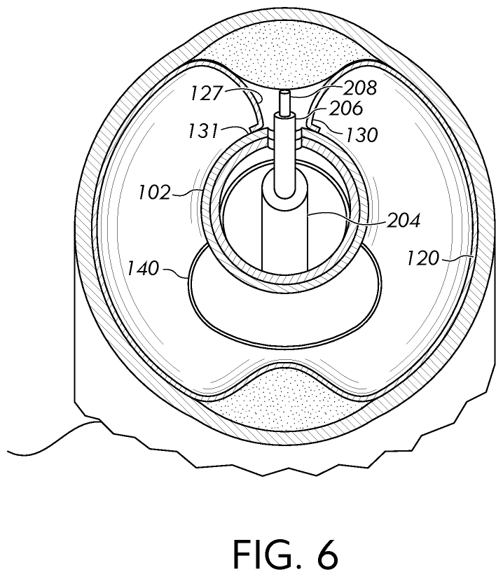

The hourglass shape of the balloon 120 enables the balloon to be positioned at a stenotic lesion or other treatment site with the treatment site generally centered at the neck 125 thereof with proximal and distal portions of the balloon in contact with healthy tissue surrounding the treatment site. In an artery having a stenotic lesion, in a deployed state as shown in FIG. 5, the proximal and distal portion of the balloon contact endothelium before and after the plaque or stenosis with a pressure suitable to support this healthy endothelium and form a seal that retains the plaque within the neck of the balloon and reduces the possible risks of plaque entering the blood stream.

The plurality of internal chambers 122 of the balloon 120 are inflatable sequentially or simultaneously and are controllably inflatable. In FIG. 2, there is a proximal chamber 122a, a neck chamber 122b, and a distal chamber 122c, but is not limited thereto. In other embodiments, the balloon can have any number of chambers from 4 to 16 or more segments. In one embodiment, there are four or more segments radially and three or more segments longitudinally, for a total of twelve or more segments in the balloon 120.

In one embodiment, represented by the dashed lines in FIG. 2, the port 130 includes a radially extendable tube 132 fixed to the neck chamber 122b. Here, inflation of the neck chamber 122b extends the tube 132 radially outward as desired based on the size of the stenotic lesion or treatment site to place the outlet 134 more proximate the treatment site. The radially extendable tube 132 can be made of balloon material. If the stenotic lesion size is large, inflatable balloon segment may be inflated proportionately to the size thereof to extend the tube 132. The length of the tube 132 may be adjusted using the additional inflation or deflation of the immediately neighboring balloon chambers.

Still referring to FIG. 2, the balloon 120 includes a radially collapsible/expandable conduit 140 juxtaposed to the elongate shaft 102, radially surrounded by the balloon 120, and having open distal and proximal ends 142, 144, when inflated, in fluid communication with the environment. This conduit 140 facilitates blood flow from proximal to distal therethrough for maintained blood flow within the vessel during any procedure, thereby functioning as an anti-infarction conduit. In other words, oxygenated blood can flow to the heart when a procedure is being performed in an artery. Since the conduit 140 is expandable, during inflation of the balloon 120, the conduit will proportionally increase in size therewith. Another advantage of the conduit 140 and maintaining blood flow during a procedure is enabling the operator to control the duration of procedure and balloon inflation/deflation, thereby enabling a longer period of time for procedures.

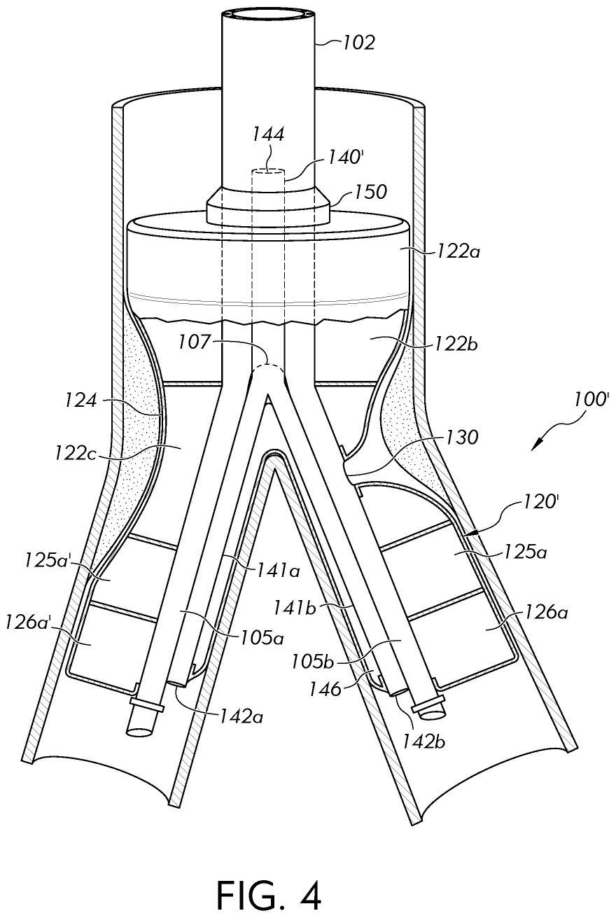

Turning now to FIG. 4, in another embodiment, the elongate shaft 102 is bifurcated at the distal end into a first hollow body 105a and a second hollow body 105b and comprises a flexible saddle 107 between and joining the first and second hollow bodies 105a, 105b. At least a most proximal of the plurality of internal chambers 122a is fully circumferential and is positioned on the elongate shaft 102 prior to the flexible saddle 107, and a port 130 is positioned in one or both of the first and second hollow bodies 105a, 105b. The port 130 can be an array of ports as described above. The radially collapsible conduit 140′ juxtaposed to the elongate shaft 102 is bifurcated to continue along each of the first and second hollow bodied 105a, 105 as a first conduit portion 141a and a second conduit portion 141b. The radially collapsible conduit 140′ has an open proximal end 144 and first and second open distal ends 142a, 142b positioned for fluid communication with the environment. The flexible saddle 107 enables the first and second hollow bodies 1051, 105b to separate from one another into a Y-shape as the balloon 120′ is inflated. The balloon portion connected to the first hollow body 142a and the balloon portion connected to the second hollow body 142b have at least two internal chambers each 125a, 126a and 125a′, 126a′. These balloon portions are less than fully circumferential. The first and second hollow bodies 142a, 142b have a saddle balloon 146 that straddles the bifurcation of the artery. The saddle balloon 146 deploys during inflation of balloon 120′ to protect the y-shaped arterial bifurcation from being traumatized by contact with any more rigid component of the balloon catheter.

In both embodiments, the balloon 120, 120′ has a length that can vary as needed for each patient, i.e., in correlation to the length of the stenotic lesion and proper placement of the proximal and distal portions of the balloon before and after the plaque. As such, the balloons 120, 120′ can be manufactured in a plurality of different custom lengths. The diameter of the balloon 120, 120′ can vary as needed for each patient, i.e., in correlation to the diameter of the artery or other vessel to receive the balloon catheter 100. In addition to the diameter of the balloon 120, 120′, the diameter of the elongate shaft 102 may vary depending upon the procedure to be carried out and the number and type of tools to be introduced to the treatment site through the port 130 in the balloon catheter 100. Example tools include, but are not limited to light, imaging, cutting, and drug deliver. Likewise, the shape and size of the port 130 can vary as well for the same reasons. In the illustrated embodiments, a single port 130 is shown, but the balloon catheter 100 is not limited thereto. In other embodiments, there can be a plurality of ports radially and/or in the axial direction within the neck of the balloon and elongate shaft and/or in the axial direction. Likewise, the elongate shaft can have one or more ports 131 alignable with any one or more of the plurality of ports 130 of the balloons 120, 120′.

The balloons 120, 120′ are made of convention, commercially available material, or herein after developed materials. The walls 123 defining the plurality of chambers 122 are waterproof and will not allow fluid to transfer to adjacent chambers. The walls areas strong or elastic as desired to maintain a pre-selected degree of rigidity at deployment. As best represented in FIG. 5, the neck 124 of the hourglass shape can have a contour that defines an annular or partially annular channel sloped to drain loose debris or vasoactive plaque material toward and into the port 130, referred to herein as a waistline drain 127 of the balloon 120.

In both embodiments, the cuff 150 operatively couples the balloon 120, 120′ to the elongate shaft 102 and may have a hollow shaft extending therefrom that carries the balloon and in which the elongate shaft 102 is received. In one embodiment, the cuff 150 is rotatable relative to the shaft 102 and/or is linearly translatable along the shaft 102 (it can be advanced and retracted) to enable an operator to position the balloon 120, 120′ selectively and accurately during deployment at a treatment site and in alignment with a port in the elongate shaft. The rotation of the cuff relative to the elongate shaft can be controlled by an electro-mechanical device or an electromagnetic force or other methods of robotic or machine learning devices operative by a user or a computer. The rotation and translation of the cuff to align the ports 130, 131 can occur before, during, or after deployment. After deployment, the balloon 120, 120′ may be rotated by deflating the balloon partially or fully and then rotating the balloon. The cuff 150 and elongate shaft 102 can include a lock to prevent the balloon 120, 120′ from being rotated while fully inflated.

The cuff 150 can enclose therein a plurality of valves, one each leading to and in fluid communication with one each of the plurality of chambers 122, 125. This fluid communication can be through independent delivery tubes. Saline or other fluid is delivered to the cuff 150 for distribution to the plurality of chambers 122, 125 for inflation thereof. Each of the plurality of valves can be electronically controlled by the operator or a computer. The cuff 150 may include a distribution chamber in fluid communication with the plurality of valves. The operator or a computer can control the inflation of any segment of the balloon with a desired amount of pressure and to a desired size (within specification limits) by controlling a respective one of the plurality of valves. The components of the cuff and/or the elongate shafts may be made of or include titanium clad material or may be polymer-based material or other suitable material.

The fluid is delivered to the cuff 150 be either a main deliver tube extending therefrom the length of the transport sheath 200 and elongate shaft 102 to the distribution chamber and or a plurality of individual deliver tubes, one each connected to a respective one of the plurality of valves. Distally, the main delivery tube or the plurality of delivery tubes have some slack therein before attaching to the cuff 150, thereby enabling rotational movement of the cuff relative to the elongate shaft 102. An electromechanical or electro-magnetic or electric or robotic system operates the valves. Sensor feedback from the valves provides operator or robot with information about inflation pressure and volume of fluid in each segment of the balloon. This information from all the valves and segments of balloon of the catheter creates a real time 3-D video display available to the medical professional. The volume and pressure delivered to each of the plurality of chambers 122, 125 of the balloon 120, 120′ are measured at the external controller or distributor system.

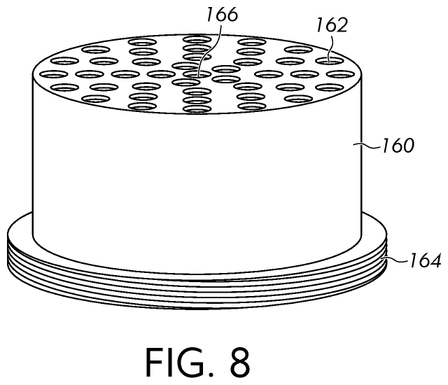

Turning back to FIG. 5 and to FIG. 8, the proximal terminal 160 of the balloon catheters 100 (or balloon catheter 100′) has a plurality of ports 162 connectable to a controller 180, which can include an inflation station 186. At least two of the plurality of ports are for control of the inflation of the balloon 120, 120′. One port is for valve control, within the cuff 150, i.e., connection to a valve controller, and the other is for pressure control of the fluid flowing to the valve, i.e., connection to/with a pressure controller. These ports connect to the inflation station 186 that is external to the patient. The inflation station 186 contains the inflation fluid and is operably by the operator or a computer/microprocessor housed therein and can include a display 182 and/or touchscreen or other input device 184, such as a keyboard, with video, audio, and controllers for all other tools that may be deployed through the balloon catheter and for operation thereof by the operator. The valve controller regulates the flow of fluid to and from each delivery tube that supplies each individual chamber of the plurality of chambers 122, 125 within the balloon 120, 120′. In one embodiment, there are as many valve controllers as there are valves.

The balloon 120, 120′ includes a means for measuring the pressure within each of the plurality of chambers 122, 125 of the balloon. The means can be any form of a sensor, such as a pressure sensor, stretch receptor (embedded in a wall of each chamber of the balloon), and/or a pressure transducer. The pressure controller 188 receives signals from the means for measuring the pressure for real time feedback and control of the pressure therein and the fluid flow from the inflation station to the valves in the cuff 150.

Turning now to FIG. 8, the connection between the terminus 160 of the balloon catheter 100, 100′ and the inflation station 180 for the valve controller 186 can have a plurality of delivery tubes arranged in an array or a single delivery tube. When a plurality of delivery tubes 162, which may be numbered to match the valves or the connection may be keyed for a single orientation for the connection. In one embodiment, a single deliver tube, each delivery tube, or the balloon catheter can have a securing mechanism 164 at the terminus thereof, such as a threaded cap, snap fit, etc. that provides a lossless connection and a fluid-tight connection. While FIG. 8 has a single threaded connection for the entire terminus of the balloon catheter, it could instead have individually threaded connections for each sheath or tube exiting the terminus and in need of connection to the inflation station. A lossless connection is one where everything going into the connection also comes out thereof, i.e., there are no losses, especially of electrical energy. In one embodiment, the connection can include a locking mechanism 166, such as a locking probe to locking receptacle configuration, often centrally positioned.

The pressure controller 188 can have a separate connection relative to the valve controller, but may be included in the terminus 160 of the balloon catheter 100, 100′. This connection may comprise a plurality of male pines and mating female sockets in any arrangement, but preferably having a single orientation for the connection to ensure the correct pin to socket are mated. The male pins and mating female sockets can be protected by an exterior surround sleeve that are mateable to one another as well.

In all embodiments, the imaging device can be any commercially available imaging device or hereinafter developed technology. In one embodiment, the imaging device is selected from near-infrared spectroscopy plus intravascular ultrasound, fiberoptics, or capacitive micromachine ultrasound transducer. Imaging will be in real time, thereby enabling the operator or computer to make decisions about balloon inflation, balloon size, and balloon positioning relative to a treatment site.

Turning now to FIG. 3, in the undeployed state, the balloon 120 and the radially collapsible conduit 140 are flat against the elongate shaft 102 and is maintained within the space defined between the elongate shaft and the outer diameter of the cuff 150. The same is true for the balloon 120′ of FIG. 4.

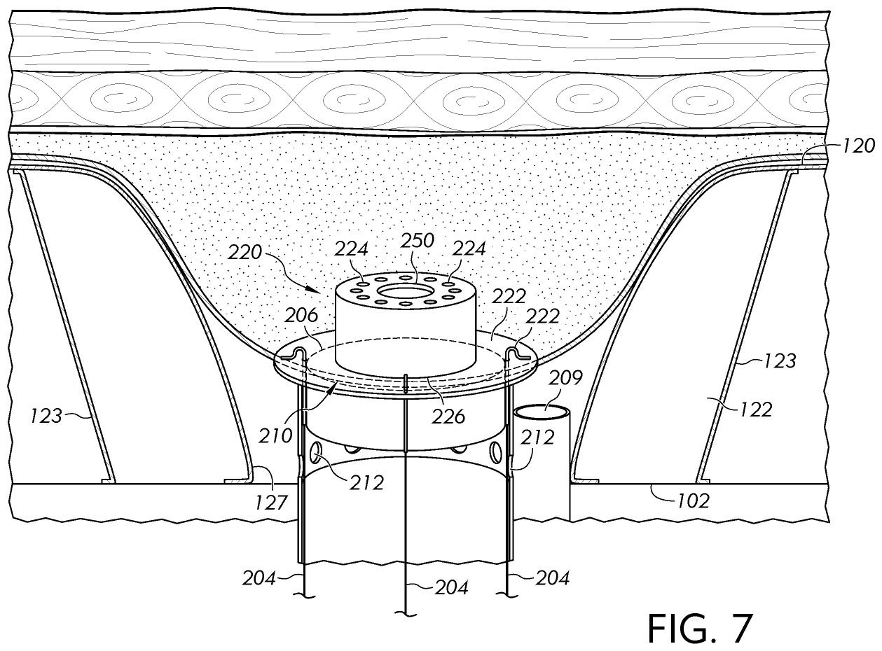

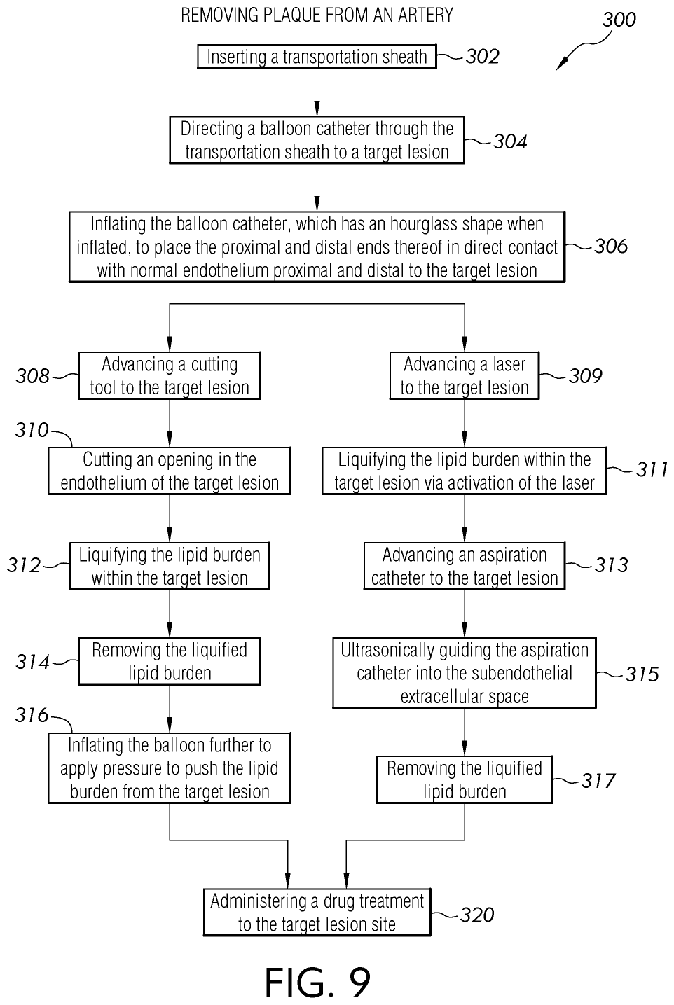

In another aspect, with reference to FIG. 9, methods of removing plaque from an artery 300 are disclosed. The methods include inserting 302 a transportation sheath 200 into an artery, directing 304 through the transportation sheath 200 a balloon catheter 100, 100′ as described herein to a target lesion in need of plaque treatment, inflating 306 the balloon 120, 120′ of the balloon catheter 100, 100′ to its generally hourglass shape (simultaneously or sequentially) with the proximal and distal ends of the balloon 120, 120′ in direct contact with normal endothelium proximal and distal to the target lesion and the neck 124 of the balloon at the target lesion, thereby isolating the target lesion from the remainder of the artery, advancing 308 a cutting tool 208 to the target lesion through the port 130 in the balloon catheter 100, cutting 310 an opening (shown in FIG. 7) into the endothelium of the target lesion, thereby creating a flap (not shown) of endothelium and access to the plaque inside the target lesion, and removing the plaque from inside the target lesion. The cutting can be a laser and the opening is a circular cut and the flap has a connection tether (an uncut region) of 25 to 45 degrees, preferably 40 degrees. The laser may be an ultraviolet laser, such as an excimer or exciplex laser. One commercially available laser is the ELCA™ laser from Phillips (FDA approved) adapted to fit the lumen utilized for delivery to the treatment site. The connection tether is at a position of upstream arterial flow, thereby arterial flow holds the flap closed after the treatment. Removing the plaque can be accomplished by applying a laser set at a preselected frequency to liquefy the lipid burden 312 present and draining 314 the liquid from the target lesion. The initial inflation of the balloon 120, 120′ can be performed in a manner that begins to push the periphery of the plaque toward the position selected for the opening. The method can also include applying increased pressure 316 to the target lesion by increasing the inflation of one or more of the plurality of internal chambers of the balloon, i.e., additional inflation of the balloon, to push plaque toward the flap opening, aspirating inside the target lesion, and closing the flap.

The method can include introduction of a tool configured to determine the lipid burden of the target lesion and using such tool to determine whether the lipid burden includes a high lipid burden, a low lipid burden, or a high to low lipid burden ratio. This introduction can include feeding the tool through the elongate shaft 102 to the port 130 and into the external environment, including inside the target lesion after the opening with flap has been cut. The tool can be near-infrared spectroscopy plus intravascular ultrasound or a capacitive micromachine ultrasound transducer, which are also capable of confirming the type of lesion, the lesion's morphology, and anatomical limits. These tools can also be used for imaging the target lesion during all steps of the medical procedure. The capacitive micromachine ultrasound transducer is much smaller than other imaging device, which can provide the benefit of being able to perform the method is smaller lumen, i.e., smaller arteries and veins.

After the imaging just described, the operator of the balloon catheter can select a point for endothelial entry (where to position the opening and flap). The balloon is positioned accordingly, with ports 130, 131 aligned, and the balloon is inflated such that the proximal and distal chambers of the balloon contact the healthy endothelium proximal and distal to the target lesion, such that the target lesion and surgical field are totally isolated from the remainder of the artery. As discuss above, when the balloon 120, 120′ is inflated, the conduit 140 is also expanded and activated to provide continuous blood flow to the distal segment of the artery to perfuse distal myocardium throughout the method. Since the conduit will restore blood flow to the myocardium that has been “starved” because of the presence of the stenotic lesion, the operator should carefully inflate the balloon at a rate that will not result in a reperfusion arrhythmia.

Additionally, the method can include administering a drug treatment to one or more of a sub-endothelium space between the endothelium and muscularis, an interior surface of the flap, and an exterior surface of the flap. The drug treatment can include collagen and/or carbon dots comprising stem cells. When collagen is present, the method includes activating the collagen by application of an activating wavelength of energy.

The stenotic lesion can be evidenced/diagnosed using any prior art techniques or hereinafter developed techniques. The insertion of the transportation sheath 200 can be accomplished using any prior art techniques or hereinafter developed techniques, such as the percutaneous approach.

With reference to FIG. 5, the tools needed for various aspects of the method can be fed through the elongate shaft 102 of the balloon catheter 100 individually or collectively in a catheter sheath 204. The catheter sheath 204 may include therein one or more aspirators, imaging device, cutting tool, and other tools needed during the medical procedure. The catheter sheath 204 is advanced to the ports 130, 131 and the tools are extended through the ports 130, 131 individually and/or collective as needed to treat the target lesion. The cutting tool is typically operated with aspiration from the aspirator 206. The aspirator may be able to rotate its head 360 degrees. The aspirator 206 can include a plurality of side ports that aspirate any possible leaks flowing through the waistline drain 127 of balloon 120, 120′.

When cutting the opening, the laser in cutting the opening, which is typically has a diameter that is equal to the diameter of the aspirator catheter plus 5%, the laser first carves out the desired degrees for the opening and flap and secondly, after switching to deeper laser, heat is applied to loosen the sub-endothelial extracellular matrix and separate the endothelium from extracellular matrix and arterial media. Gentle saline flush is applied, and the laser is used to gently move the endothelial flap away from the plaque. The laser can then be withdrawn in preparation of entering the endothelial space. Gentle aspiration is continuously applied by the aspirator to assure that no sub-endothelial plaque material escapes. Any escaping fluid will be trapped and aspirated by the drain ports 212, see FIG. 7.

Next, still referring to FIG. 7, the catheter sheath 204 is slowly advanced through the opening 210 and carries with it the aspirator 206. The catheter sheath's advancement is stopped at a position to provide a gap sufficient for a laser assembly 220 to be deployed through the catheter sheath 204. Imaging can be used to assist the operator is proper positioning and advancement thereof. The laser assembly 220 is deployed such that the entire diaphragm 222 thereof is fully opened. The aspirator catheter is then gently retracted until the laser assembly's diaphragm rests snugly on sub-endothelial lining inside the wall of the artery. A slight retracting tug is applied to ensure a coning effect thereby ensuring a funnel shape to the diaphragm to facilitate drainage of extracellular matrix liquid and debris to the aspirator 206. The aspirator 206 is then secured in this position with a locking system controlled by the operator or a computer system.

Once set and verified by the imaging device 209, the laser is set to an appropriate frequency to liquefy the lipids inside the stenotic lesion. The laser may be retractable, for example, it may be spring loaded 226 for maintaining contact with the plaque. As discussed above, an excimer or exciplex laser is selected depending upon the plaque material, i.e., whether it is high lipid burden or low lipid burden. In one embodiment, the plaque material is or has a high lipid burden and the laser is a 635 nm, 10-mW diode excimer laser. The laser is applied for about 6 minutes to the plaque material. During application of the laser, the laser or the catheter sheath 204 (including the laser) is rotated through a 360 degree turn to ensure all plaque material is being liquified. The transitory pores in the cell membrane of adipocytes open and 99% of fat is released from the adipocytes. All vasculature and extracellular matrix structure should remain unharmed by this laser energy. The aspirator 206 is used as needed to remove debris and the balloon is controllably inflated as needed to ensure that significant portion of adipocytes are exposed to the laser energy and the liquefied fat is being “milked” toward the aspirator 206 and drain ports 212. In other words, the plaque will be liquified or vaporized by the laser and aspirated (sucked) out by the aspirator 206. No material should leak outside into the lumen of the artery due to the diaphragm 222 that seals the opening 210. As the medical procedure continues, the neck chamber of the balloon 120, 120′ can be inflated to have a diameter similar to that of the other plurality of chambers to restore the artery lumen to near its original normal size. However, inflation is stopped to leave a layer of space in the extracellular matrix between the endothelium and the muscularis layer of the artery. The muscularis layer and the artery wall does not receive any excess pressure or damage from laser or aspiration and thus remains intact and unstretched or injured. Using the imaging device, the operator can confirm that all the plaque material has been removed and the lumen of the artery is fully or almost restored to normal size.

The method includes closing the flap once the plaque has been removed. The method can also include administering a drug treatment to one or more of a sub-endothelium space between the endothelium and muscularis, on an interior surface of the flap, and on an exterior surface of the flap. Of these treatments, only the application to the exterior surface of the flap is performed after the flap is closed. In one embodiment, the drug treatment comprises collagen and/or carbon dots comprising stem cells. When collagen is present, the collagen can be activated by application of an activating wavelength of energy. Any suitable tool can be fed through the elongate shaft to the ports 130, 131 to deliver the drug treatment. In one embodiment, the drug treatment is in the form of a patch. The patch dispenses medications that aid in healing the endothelium at the flap site. In one embodiment, the medication comprises undifferentiated stem cells, such as mesenchymal stem cells. The stem cells differentiate into a single layer endothelial cell and provide a seamless healing of the flap in less than 24 hours.

Referring back to FIG. 7, the laser assembly 220 may include a delivery port 250, which is shown in the center thereof, but is not limited to that position. In other, embodiments, the laser may be retracted and a delivery catheter may be introduced through the aspirator to deliver a drug treatment to the treatment site. In one embodiment, the drug treatment is collagen and carbon dot tagged mesenchymal stem cells pharmaceutically effective amounts. The drug treatment may be perfused into the sub-endothelial space (extracellular matrix) so as to evenly spread between the endothelium and the muscularis. The mesenchymal stem cells can be pre-conditioned with ET-1 to facilitate their survival and activation. Specific wavelength 210 nm emitting tip, such as an ALN LED or Aluminum Nitride LED, is introduced to activate the release of the collagen. This released collagen will lay evenly on the surface of the extracellular matrix because of the gentle even pressure applied by the balloon. The LED wavelength may also dissociate the stem cells, which will differentiate into functional endothelium to provide a resupply of endothelial cells. Then, a deep ultraviolet scan can be performed by the LED tip to confirm adequacy of drug treatment, i.e., evidence of no leaks from the extracellular matrix. Once confirmed, the tools are withdrawn from the target lesion.

Next, if desired, another patch can be applied to the inside surface of the endothelial flap before the flap is moved to a closed position. The patch can again include a drug treatment of collagen and carbon dot tagged mesenchymal stem cells in pharmaceutically effective amounts. Activation by 210 nm wavelength LED is again appropriate for the same reasons noted above. The flap is moved to the closed position by an appropriate tool. The collagen will keep the flap adhered to sub-endothelial tissue, and the endothelium will reorganize over a period of days, thereby restoring the lumen.

Once the flap is closed, another patch can be applied to the exterior surface of the flap using an appropriate tool. The patch can again include a drug treatment. Here, the drug treatment can include carbon dots, clopidogrel, and sirolimus in pharmaceutically effective amounts. In one embodiment, the drug treatment includes 1.875 mg of clopidogrel and 140 micrograms of sirolimus. This patch is preferably at least 50% larger than the flap for adequate protection of the normal adjacent endothelium. Hereto, activation by 210 nm wavelength LED is appropriate. The clopidogrel and sirolimus are activated for a time-release by the LED. Clopidogrel will prevent platelet aggregation at the site of endothelial window (similar to a drug eluting stent) and sirolimus will prevent excessive endothelial proliferation in the lumen of the artery.

Referring again to FIG. 9, in another embodiment, the method of removing plaque from an artery begins similarly with the introduction of a transportation sheath and insertion of one of the balloon catheters described herein. Once the balloon is inflated at the treatment site, a LASER appropriate for the lipid burden is placed adjacent to the plaque lesion 309, i.e., the arterial luminal endothelial surface of the artery, and activated 311 without entry into the subendothelial space to liquefy the lipid. The liquified lipid is released into the subendothelial extracellular matrix. When the lipid burden is high, a 635 nm LASER is used. When the lipid burden is low and calcium burden is high, a higher frequency (680 nm) LASER is used. On a scale, increasing concentration or ratio of lipid (to calcium) results in decreasing the frequency of the LASER because lower intensity LASER at 635 nm is ideal for adipocytes to open pores and release 90% of the lipid material in a liquified state. Decreasing ratio of lipid to calcium requires higher LASER frequency to breakdown calcium and allow adipocytes to release the calcium/lipid liquified material through the pores.

Then, an aspiration catheter is ultrasonically guided 313, 315 into the subendothelial extracellular matrix space and the liquified fatty material is aspirated 317 therefrom. The aspirator catheter may be ultrasonically guided into the subendothelial space before or after the activation of the laser. The benefit of this alternate method is that no window must be cut in the endothelial lining. After removal of the liquified lipid, a drug treatment may be administered 320 the subendothelial space between the endothelium and muscularis in one or more locations in or near the target lesion. The drug treatment can include collagen and/or carbon dots comprising stem cells. When collagen is administered, the method includes activating the collagen and/or carbon dots by application of activating wavelength energy. In one embodiment, the drug treatment is a collagen-carbon dot complex.

After either of the methods discussed above are completed, administration of a drug treatment(s) to the target lesion site 320 occurs and thereafter the tools, balloon catheter, and transportation sheath are removed from the patient. Following the medical procedure, the medical professional may instruct the patient to take baby aspirin (81 mg), one per day, thereafter for a pre-selected time period or for the life of the patient. Plavix oral therapy daily may be prescribed as well for a selected time period, such as 3 months when a stent is not deployed or one year when a stent is deployed.

Multiple advantages result from use of the balloon catheter and methods of treatment therewith. The primary goals being recanalization of a stenotic vessel, preventing endothelial and arterial damage, and reducing the plaque burden without spreading it into adjacent normal artery wall. Controlled inflation of the balloon catheter into its plurality of chambers reduces the risk of damage to the blood vessel or its internal structure, aneurysm, dissection, and collapse of an artery. The controlled inflation also provides control over inflation at a bend in a vessel or an occlusion at a fork or branch origin of a segmental artery. The balloon catheter disclosed herein, in contrast to conventional balloon catheter technology, reduces deformation and degree of tensile strength applied on the overall artery by inflation at lower amounts of cumulative pressure against the arterial internal lining (intima).

The medical procedure disclosed herein should eliminate the use of a stent in most instances, and when a stent needs to be deployed, both minimum stent material and minimum pressure will be needed in the deployment. When stenting is unavoidable, a protective layer for the endothelium will be created and placed through nanotechnology. This will both limit endothelial damage and prevent damage to the original endothelium. Commercially available biodegradable stents, which gradually degrade and gets absorbed in the body or eliminated by excretion of the biodegradable materials, may also be deployed after the medical procedure disclosed herein.

The balloon catheters disclosed herein and the various methods of use of such balloon catheters can be implements to treat various conditions of the coronary artery, such as but not limited to: atheromatous sub-total occlusion; critical stenosis; and atheromatous plaque stenosis; atheromatous sub-total occlusion; critical stenosis of the carotid artery; critical (sub-total) or non-critical renal artery stenosis; femoral and popliteal artery atheromatous stenosis; ileo-femoral atheromatous stenosis; aorto-Iliac atheromatous stenosis; atheromatous pulmonary artery stenosis; and intra-cerebral atheromatous sub-critical stenosis. When the balloon is bifurcated, treatments can include bifurcation coronary artery stenosis in left main coronary artery, left anterior descending artery, or left circumflex artery, or bifurcation coronary artery stenosis in the bifurcation aorto-iliac stenotic lesions, bifurcation ileo-femoral stenotic lesions, and bifurcation femoral-popliteal lesions.

Although the invention is shown and described with respect to certain embodiments, modifications will occur to those skilled in the art upon reading and understanding the specification, and the present invention includes all such modifications.

What is claimed is:

1. A balloon catheter comprising:
an elongate shaft having an internal hollow body; and
a balloon at the distal end of the shaft, the balloon being inflatable and deflatable in accordance with the pressure of a fluid supplied to the inside of the balloon through the internal hollow body of the shaft;
the balloon comprising:
a plurality of internal chambers that are inflatable to differing pressures, thereby, when inflated, the balloon has a generally hourglass shape having a neck between a distal end and a proximal end of the balloon; and
a port at the neck of the balloon in open communication with a delivery tube housed within the internal hollow body and in open communication with an environment external to the balloon.

2. The balloon catheter of claim 1, wherein the plurality of internal chambers of the balloon are inflated sequentially or simultaneously.

3. The balloon catheter of claim 2, wherein the plurality of internal chambers are controllably inflatable.

4. The balloon catheter of claim 3, wherein the plurality of internal chambers comprise at least a distal chamber, a neck chamber, and a proximal chamber.

5. The balloon catheter of claim 4, wherein the port includes a radially extendable tube fixed to the neck chamber; wherein inflation of the neck chamber extends the tube radially outward.

6. The balloon catheter of claim 1, comprising a radially collapsible conduit juxtaposed to the elongate shaft, radially surrounded by the balloon, and having open distal and proximal ends, when inflated, in fluid communication with the environment.

7. The balloon catheter of claim 1, wherein the elongate shaft is bifurcated at the distal end into a first hollow body and a second hollow body and comprises a flexible saddle between and joining the first and second hollow bodies; wherein at least a most proximal of the plurality of internal chambers is fully circumferential and is positioned on the elongate shaft prior to the flexible saddle, and the port is positioned in one of the first and second hollow bodies.

8. The balloon catheter of claim 7, comprising a radially collapsible conduit juxtaposed to the elongate shaft that is bifurcated to continue along each of the first and second hollow bodied; wherein the radially collapsible conduit has an open proximal end and first and second open distal ends positioned for fluid communication with the environment.

9. The balloon catheter of claim 8, wherein flexible saddle enables the first and second hollow bodies to separate from one another into a Y-shape as the balloon is inflated.

10. The balloon catheter of claim 9, wherein the first balloon portion connected to the first hollow body and the second balloon portion connected to the second hollow body comprise at least two internal chambers each; wherein the first balloon portion and the second balloon portion are less than fully circumferential.

11. The balloon catheter of claim 1, comprising a cuff operatively connecting the balloon to the elongate shaft; wherein the cuff is rotatable relative to the elongate shaft.

12. The balloon catheter of claim 11, wherein the cuff is linearly translatable along the elongate shaft.

13. The balloon catheter of claim 11, wherein the balloon in a deflated state lay inside an outer perimeter defined by the cuff.

14. A method of removing plaque from a lesion in a lumen of a patient in need thereof, the method comprising:
deploying a balloon catheter according to claim 1 to a target lesion in need of plaque treatment;
inflating the balloon of the balloon catheter to its generally hourglass shape with the proximal and distal ends of the balloon in direct contact with normal endothelium proximal and distal to the target lesion and the neck of the balloon at the target lesion, thereby isolating the target lesion from the remainder of the artery;
liquifying the plaque; and
removing the plaque from inside the target lesion.

15. The method of claim 14, comprising additional inflation of the balloon to push plaque toward the flap opening.

16. The method of claim 14, comprising determining the lipid burden of the plaque.

17. The method of claim 16, wherein determining the lipid burden includes application of near-infrared spectroscopy plus intravascular ultrasound or a capacitive micromachines ultrasound transducer.

18. The method of claim 14, wherein the method further includes advancing a cutting tool to the target lesion through the port in the balloon catheter; cutting an opening into the endothelium of the target lesion, thereby creating a flap of endothelium and access to plaque inside the target lesion.

19. The method of claim 18, wherein the cutting tool is a laser and the opening is a circular cut and the flap has a connection tether of 25 to 45 degrees.

20. The method of claim 19, wherein the connection tether is at a position of upstream arterial flow, thereby arterial flow holds the flap closed subsequent to the treatment.

21. The method of claim 19, wherein removing the plaque comprises applying a laser set at a preselected frequency to liquefy the lipid burden present, and draining the liquid from the target lesion.

22. The method of claim 21, comprising aspirating inside the target lesion.

23. The method of claim 21, comprising closing the flap.

24. The method of claim 21, comprising administering a drug treatment to one or more of a sub-endothelium space between the endothelium and muscularis, an interior surface of the flap, or an exterior surface of the flap.

25. The method of claim 24, wherein the drug treatment comprises collagen and/or carbon dots comprising stem cells.

26. The method of claim 25, comprising activating collagen by application of an activating wavelength of energy.
